(19) United States
(12) Patent Application Publication
(10) Pub. No.: US 2015/0011859 A1
(43) Pub. Date: Jan. 8, 2015

(54) ELASTIC MODULUS MEASURING APPARATUS AND ELASTIC MODULUS MEASURING METHOD

(71) Applicant: Kabushiki Kaisha Toshiba, Tokyo (JP)

(72) Inventors: Motomichi Shibano, Atsugi-Shi (JP); Mineharu Uchiyama, Kawasaki-Shi (JP); Hiromichi Kobori, Yokohama-Shi (JP)

(73) Assignee: Kabushiki Kaisha Toshiba, Tokyo (JP)

(21) Appl. No.: 14/167,432

(22) Filed: Jan. 29, 2014

(30) Foreign Application Priority Data
Jul. 3, 2013 (JP) 2013-140068

(51) Int. Cl. A61B 5/00

(52) U.S. Cl. CPC A61B 5/0095; A61B 5/442
USPC 600/407

(57) ABSTRACT

An elastic modulus according to the present embodiment includes a light source configured to generate light having a wavelength at which a material within a measurement target absorbs the light. An optical system causes the light to be passed through the measurement target at a desired aperture diameter and focuses the light on the material. A detector contacts the measurement target and detects an acoustic wave generated when the material absorbs the light. An operation unit calculates an elastic modulus of the material using a first acoustic-wave measurement value obtained in a case where the aperture diameter is a first diameter and a second acoustic-wave measurement value obtained in a case where the aperture diameter is a second diameter different from the first diameter.

### CROSS REFERENCE TO RELATED APPLICATIONS

This application is based upon and claims the benefit of priority from the prior Japanese Patent Application No. 2013-140068, filed on Jul. 3, 2013, the entire contents of which are incorporated herein by reference.

### FIELD

The embodiments of the present invention relate to an elastic modulus measuring apparatus and an elastic modulus measuring method.

### BACKGROUND

Conventionally, such techniques as suction, traction/compression, Ballistometry, ultrasonic vibration, torsion, and wave propagation have been invented so as to measure the elastic modulus (softness or elasticity) of a skin without damaging the skin.

However, the conventional techniques have the following problems. The skin has a multilayer structure including the epidermis, the dermis, the subcutis, and the like having different elastic modulus. With the conventional techniques, it is disadvantageously difficult to measure the elastic modulus of each layer because the different layers influence one another at the time of the measurement.

### BRIEF DESCRIPTION OF THE DRAWINGS

FIG. 1 shows an example of a configuration of an elastic modulus measuring apparatus 1 according to a first embodiment;

FIG. 2 is a flowchart showing an elastic modulus measuring method according to the first embodiment; and

FIG. 3 shows a state where the elastic modulus measuring apparatus 1 measures the elastic modulus of the skin

### DETAILED DESCRIPTION

Embodiments will now be explained with reference to the accompanying drawings. The present invention is not limited to the embodiments.

An elastic modulus according to the present embodiment includes a light source configured to generate light having a wavelength at which a material within a measurement target absorbs the light. An optical system causes the light to be passed through the measurement target at a desired aperture diameter and focuses the light on the material. A detector contacts the measurement target and detects an acoustic wave generated when the material absorbs the light. An operation unit calculates an elastic modulus of the material using a first acoustic-wave measurement value obtained in a case where the numerical aperture diameter is a first diameter and a second acoustic-wave measurement value obtained in a case where the numerical aperture diameter is a second diameter different from the first diameter.

### First Embodiment

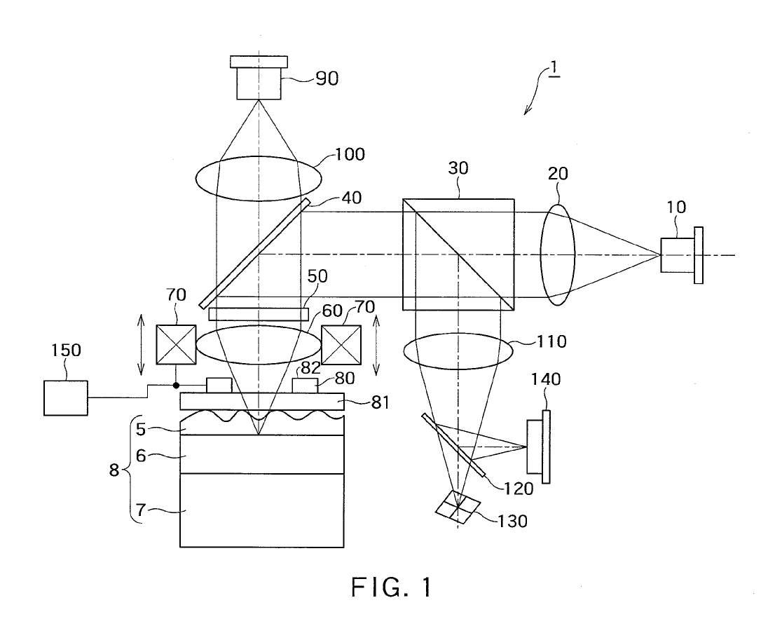

FIG. 1 shows an example of a configuration of an elastic modulus measuring apparatus 1 according to a first embodiment. The elastic modulus measuring apparatus 1 includes a near-infrared laser diode 10 that serves as a light source, a collimator lens 20, a beam splitter 30, a reflecting mirror 40, an aperture adjustment unit 50, an objective lens 60, a focus adjustment unit 70, a microphone 80, a white LED (Light Emission Diode) 90, a collimator lens 100, an adjustment lens 110, a beam splitter 120, a quadrant PD (Photo Detector) 130 that serves as a focus detection element, a CMOS image sensor 140, and an operation control unit 150.

For example, a human skin or an animal skin can be used as a skin 8. The skin 8 has a multilayer structure including an epidermis 5, a dermis 6, and a subcutis 7 (hereinafter, also simply “layer 5”, “layer 6”, and “layer 7”, respectively). The elastic modulus measuring apparatus 1 is used to calculate an elastic modulus of each of the layers 5, 6, and 7 of the skin 8. Therefore, the elastic modulus measuring apparatus 1 is applicable for such purposes as medical devices, beauty appliances, and health appliances. Because the skin 8 contains much water inside, the elastic modulus measuring apparatus 1 can stably measure the elastic modulus.

The near-infrared laser diode 10 emits light under control of an electric signal transmitted from an external drive circuit (not shown). The near-infrared laser diode 10 (hereinafter, also “LD 10”) emits near-infrared laser light having a wavelength of 1.45 μm so that the water contained in the skin 8 can absorb light energy. That is, the LD 10 generates the near-infrared laser light having the wavelength at which a material within the skin 8 can absorb the near-infrared laser light. Such near-infrared laser light generates an photoacoustic effect within the skin 8.

Laser light is pulsed light emitted in a certain cycle (at a certain frequency). When the laser light condenses on a predetermined focal position within the skin 8, then water at the focal position absorbs the laser light, and the water is instantaneously adiabatically expanded. An acoustic wave is generated by such expansion of the water within the skin 8. The acoustic wave is generated whenever the skin 8 is irradiated with a laser light pulse.

The collimator lens 20 collects the laser light from the LD 10 and converts the laser light into collimated laser light. The beam splitter 30 has character, for example, to transmit 90% or more of laser light and to reflect 90% or more of visible light. Therefore, the beam splitter 30 transmits most of the laser light from the collimator lens 20 and reflects most of visible light from the reflecting mirror 40.

The reflecting mirror 40 has character, for example, to reflect 90% or more of laser light and to transmit 90% or more of visible light. Therefore, the reflecting mirror 40 reflects most of the laser light from the beam splitter 30 to the skin 8 and transmits most of visible light from the white LED 90 toward the skin 8.

The aperture adjustment unit 50 includes an aperture that allows the laser light or the visible light from the reflecting mirror 40 to partially pass through. That is, the aperture adjustment unit 50 allows the laser light from the LD 10 to pass through at a desired aperture diameter to the skin 8. For example, a transmissive liquid crystal device controlling transmittance of the laser light can be used as the aperture adjustment unit 50. The aperture adjustment unit 50 determines a numerical aperture NA by adjusting an aperture diameter DA of the aperture. The numerical aperture NA is a value obtained by dividing a half of the aperture diameter DA (an aperture radius) by a focal length of the objective lens 60.

The objective lens 60 collects the laser light or the visible light passed through the aperture adjustment unit 50 and focuses the laser light within the skin 8. The focus adjustment unit 70 is provided at the objective lens 60, and the focus adjustment unit 70 controls a position of the objective lens 60 to be close to or away from the skin 8. The objective lens 60 can thereby focus (adjust a focus of) the laser light on an arbitrary position in a range from the epidermis 5 to the subcutis 7 of the skin 8. The focus adjustment unit 70 is constituted by a voice coil, a lens holding member, and a drive circuit (not shown) and changes a focal position of the laser light dependently on an amount of current from the drive circuit.

The microphone 80 includes a glass substrate 81 transmitting laser light and visible light and a plurality of highly sensitive silicon microphones 82 detecting an acoustic wave. The glass substrate 81 contacts a surface of the skin 8 so as to detect the acoustic wave generated within the skin 8. The highly sensitive microphones 82 detect the acoustic wave propagated from the skin 8 via the glass substrate 81. Examples of types of the highly sensitive microphones 82 include a MEMS (Microelectromechanical System) microphone, a capacitor microphone, and a piezoelectric effect microphone. In the present embodiment, MEMS microphones are used as the highly sensitive microphones 82. Each of the MEMS microphones includes a diaphragm formed on a silicon substrate (not shown) vibrating in response to an acoustic wave and converts a vibration of the diaphragm into an electric signal to detect the acoustic wave. By using the MEMS microphones as the highly sensitive microphones 82, the highly sensitive and small-sized microphone 80 can be provided. A window region is provided in the glass substrate 81 so as not to obstruct the laser light and white light. The highly sensitive silicon microphones 82 are arranged on the glass substrate 81 so as not to overlap the window region but to be proximate to the window region. The highly sensitive silicon microphones 82 are arranged uniformly around the window region so as to be able to accurately measure the acoustic wave. For example, the window region is a circular region having a diameter of 1 mm, and the highly sensitive silicon microphones 82 are arranged circularly to surround the window region. By thus uniformly arranging the highly sensitive silicon microphones 82, the microphone 80 can detect the acoustic wave with high sensitivity.

The white LED 90 is provided to observe the surface of the skin 8. The white LED 90 emits visible light under control of an electric signal from an external drive circuit (not shown). The white LED 90 (hereinafter, also “LED 90”) emits white light as the visible light. The collimator lens 100 converts the white light into collimated light. The white light from the collimator lens 100 is transmitted through the reflecting mirror 40 and the skin 8 is irradiated with the while light. The white light is reflected from the surface of the skin 8, and reflected by the reflecting mirror 40 and the beam splitter 30 via the objective lens 60 and the aperture adjustment unit 50.

The adjustment lens 110 collects the light from the beam splitter 30. The beam splitter 120 reflects the white light from the adjustment lens 110 on the CMOS image sensor 140. The CMOS image sensor 140 receives the white light and picks up an image of the surface of the skin 8. The CMOS image sensor 140 displays the surface image of the skin 8 on a display device (not shown). It is thereby possible to observe the skin 8 on a uniform skin surface with fewer variations by avoiding regions inappropriate for measurement, for example, where injuries, moles, or pores are present.

In this example, an optical axis of the white light from the LED 90 to the skin 8 coincides with that of the laser light from the reflecting mirror 40 to the skin 8. It is thereby possible to observe the surface of the skin 8 on which the laser light condenses by observing the white light reflected from the skin 8. That is, a user can easily confirm a position of the skin 8 irradiated with the laser light by referring to the display device.

The quadrant PD 130 is provided to measure the focal position of the laser light. That is, the quadrant PD 130 receives the laser light or the visible light reflected from the skin 8 and detects whether the laser light is focused on a desired region within the skin 8. For example, the quadrant PD 130 detects the laser light reflected and returned from the skin 8. As described above, the beam splitter 30 reflects a part of the laser light on the quadrant PD 130 while transmitting about 90% of the laser light. The quadrant PD 130 detects this part of the laser light. The quadrant PD 130 then detects the focal position using the laser light in which astigmatism is generated. A focal position signal is transmitted from the quadrant PD 130 to an external control circuit (not shown) and used for controlling the focal position. The focal position cannot be accurately adjusted because of light scattering within the skin 8. For this reason, the focal position within the skin 8 is adjusted based on a boundary between the skin 8 and the glass substrate 81. Alternatively, the quadrant PD 130 can detect the focal position using the visible light. In this way, use of the quadrant PD 130 facilitates correcting the focal position of the laser light. In another alternative, LD light can be selectively captured by providing an LED-light blocking filter on a surface of the quadrant PD 130. This can further improve measurement accuracy.

The beam splitter 120 is inclined at an angle of about 45 degrees with respect to an optical axis of the visible light incident on the CMOS image sensor 140 and an optical axis of the visible light incident on the quadrant PD 130. The beam splitter 120 causes the visible light from the adjustment lens 110 to be reflected or transmitted to both the CMOS image sensor 140 and the quadrant PD 130.

The operation control unit 150 controls the constituent elements of the elastic modulus measuring apparatus 1, and calculates the elastic modulus based on an acoustic-wave measurement value transmitted from the microphone 80. Calculation of the elastic modulus is described later.

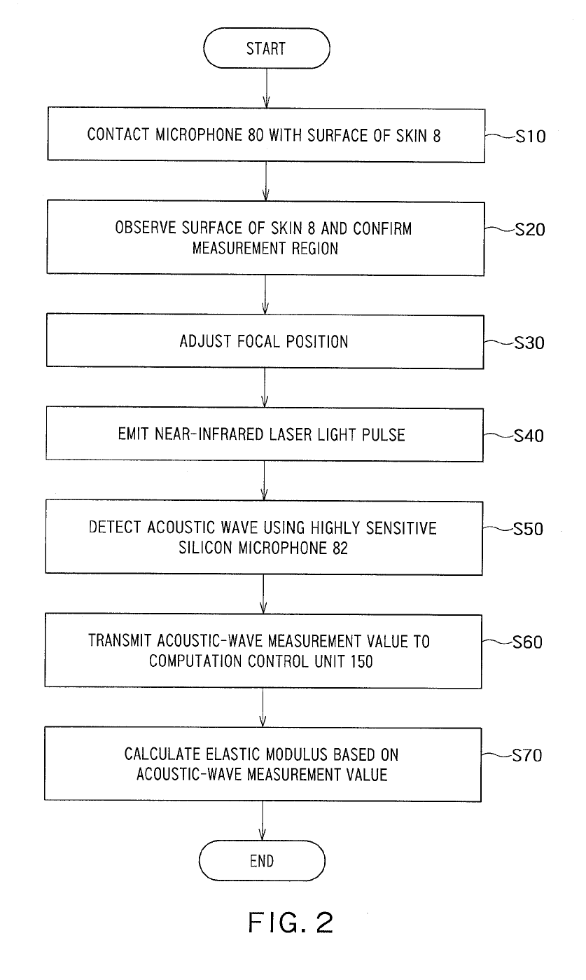

FIG. 2 is a flowchart showing an elastic modulus measuring method according to the first embodiment.

Operations performed by the elastic modulus measuring apparatus 1 and the elastic modulus measuring method are described with reference to FIG. 2.

First, an operator contacts the microphone 80 with a surface of the skin 8 (S10). The operator observes the surface of the skin 8 using the CMOS image sensor 140 and confirms a measurement region (S20). For example, in a case of measuring the elastic modulus of the normal healthy skin 8, the operator selects a region where injuries, moles, or the like that possibly obstruct the measurement are not present as the measurement region.

The LD 10 emits laser light at such a light amount that the laser light does not damage the skin 8 and irradiates the skin 8 with the laser light. The focal position of the objective lens 60 is adjusted using the quadrant PD 130 and the focus adjustment unit 70 (S30). The focal position can be automatically adjusted by causing the operation control unit 150 to control the focus adjustment unit 70 based on a detection result from the quadrant PD 130. At this time, the focal position is adjusted based on a contact surface (a boundary surface) between the glass substrate 81 and the skin 8.

Next, the LD 10 emits a near-infrared laser light pulse having a predetermined energy (S40). The objective lens 60 collects the laser light via the beam splitter 30, the reflecting mirror 40, and the aperture adjustment unit 50 shown in FIG. 1 and focuses the laser light on the focal position within the skin 8. The laser light focused on the focal position by the objective lens 60 is absorbed by water present at the focal position within the skin 8. The water is thereby adiabatically expanded and the acoustic wave is generated. For example, a spot radius of the objective lens 60 is about 3.4 μm and a focus depth is about 5 μm. An optical path length (a measurement length) of the measurement region that absorbs the laser light is normally about twice as large as the focus depth. Therefore, in the above specific example, the measurement length is, for example, about 10 μm. That is, according to the present embodiment, the acoustic wave can be generated in a restricted narrow region within the skin 8.

The microphone 80 detects the acoustic wave generated within the skin 8 using the highly sensitive silicon microphones 82 (S50). Furthermore, the microphone 80 converts the acoustic wave into an electric signal and transmits the electric signal to the operation control unit 150. That is, the microphone 80 transmits the acoustic-wave measurement value to the operation control unit 150 (S60).

The operation control unit 150 calculates the elastic modulus based on the acoustic-wave measurement value (S70).

By repeatedly performing Steps S10 to S70 while the focus position adjustment unit 70 changes the focal position, the elastic modulus measuring apparatus 1 can measure elastic moduli at various positions within the skin 8.

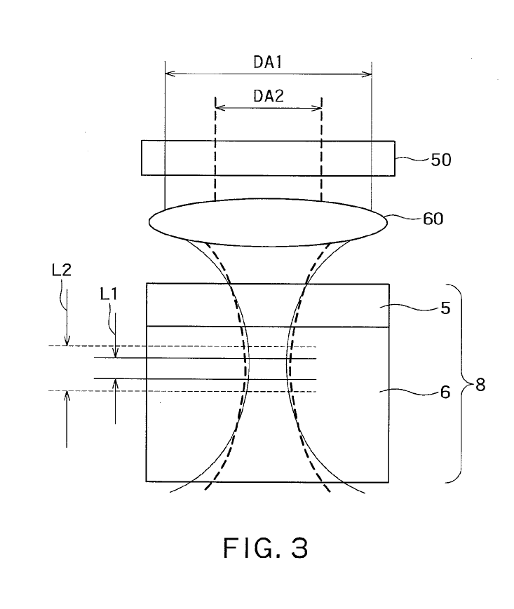

FIG. 3 shows a state where the elastic modulus measuring apparatus 1 measures the elastic modulus of the skin 8. The method of calculating the elastic modulus is described in more detail with reference to FIG. 3.

The operation control unit 150 receives a first acoustic-wave measurement value P1 (hereinafter, also “measurement value P1”) obtained in a case where the aperture diameter of the aperture adjustment unit 50 is a first diameter DA1, from the microphone 80. The operation control unit 150 receives a second acoustic-wave measurement value P2 (hereinafter, also “measurement value P2”) obtained in a case where the aperture diameter of the aperture adjustment unit 50 is a second diameter DA2 different from the first diameter DA1, from the microphone 80. The operation control unit 150 calculates the elastic modulus of the measurement region within the skin 8 using the first and second acoustic-wave measurement values P1 and P2. The measurement value P1 or P2 indicates a pressure (an acoustic pressure) obtained by the acoustic wave.

More specifically, the operation control unit 150 computes simultaneous equations obtained by applying the following Equation (1) to the case where the aperture diameter of the aperture adjustment unit 50 is the first diameter DA1 and applying the following Equation (1) to the case where the aperture diameter is the second diameter DA2. The elastic modulus (represented by B in the Equation (1)) and a light absorption coefficient (represented by β in the Equation (1)) of the measurement region within the skin 8 are thereby calculated.

$$P = - B \cdot α t \cdot \frac{{I}_{0} ( 1 - {e}^{- β L} )}{Cp \cdot L \cdot ρ \cdot f} \qquad ( 1 )$$

In the Equation (1), P represents an acoustic-wave measurement value (an acoustic pressure: Pa), B represents a bulk modulus (of elasticity) (Pa) of the measurement region, and αt represents a linear expansion coefficient (1/K) of a main material that mainly constitutes the measurement region. I_{O} represents a light intensity (W/m²) of the laser light, and β represents a light absorption coefficient (1/m) of the measurement region. L represents a measurement length (m) of the measurement region, f represents a pulse frequency (1/s) of the laser light, and ρ represents a density (kg/m³) of the main material that mainly constitutes the measurement region. Furthermore, Cp represents a specific heat at constant pressure (J/(kg×K)) of the main material that mainly constitutes the measurement region.

The acoustic-wave measurement value P is obtained at Steps S10 to S70 described above. The light intensity I_{O} is also obtained by the actual measurement. The pulse frequency f is confirmed from a setting of a drive cycle of the LD 10. The measurement length L can be calculated based on the numerical aperture NA because the measurement length L can be approximated to (the laser light wavelength)/(2·NA²). The numerical aperture NA is proportional to the aperture diameter DA because the numerical aperture NA is represented as (the aperture radius)/(the focal length) as described above. Therefore, when the aperture diameter DA is relatively large, the optical path length (the measurement length L) of the laser light is relatively small. On the other hand, when the aperture radius DA is relatively small, the optical path length (the measurement length L) of the laser light is relatively large. As shown in FIG. 3, when it is assumed that a measurement length in the case where an aperture diameter is DA1 is L1 and that a measurement length in the case where an aperture diameter is DA2 is L2, the measurement length L2 in the case where the aperture diameter is DA2 is larger than the measurement length L1 in the case where the aperture diameter is DA1.

The main constituent of the measurement region within the skin 8 used as a measurement target in the present embodiment is the water. Therefore, αt can be approximated to the linear expansion coefficient of the water (55.8/K), ρ can be approximated to the density of the water (10¹² kg/m³), and Cp can be approximated to the specific heat at constant pressure of the water (4.18 J/kg/K (at 40° C.)). Needless to mention, these parameters αt, ρ, and Cp can be changed dependently on constituent materials of the measurement target because the main constituent differs among measurement targets.

On the other hand, the bulk modulus B and the light absorption coefficient β largely change depending on an amount of the water in the skin 8. The bulk modulus B and the light absorption coefficient β differ among the layers 5 to 7 because the layers 5 to 7 differ in the amount of the water in the skin 8. Therefore, in the present embodiment, the simultaneous equations are obtained by measuring acoustic waves a plurality of times while changing the measurement length L. The operation control unit 150 calculates the bulk modulus B and the light absorption coefficient β by solving the simultaneous equations. That is, the operation control unit 150 can obtain the bulk modulus B and the light absorption coefficient β by computing the simultaneous equations as follows because the parameters other than the bulk modulus B and the light absorption coefficient β among those in the Equation (1) are already known by actual measurement, approximation, or setting.

(Case where Aperture Diameter is DA1)

In the case where the aperture diameter is DA1, the first acoustic-wave measurement value P1, a first light intensity I1, and the first measurement length L1 are substituted for the parameters in the Equation (1). The first acoustic-wave measurement value P1 and the first light intensity I1 are actually measured. The first measurement length L1 is calculated based on the aperture diameter DA1. For example, when the aperture diameter DA1 is 0.26, the first measurement length L1 is 20 μm.

By substituting the first acoustic-wave measurement value P1, the first light intensity I1, and the first measurement length L1 for the parameters in the Equation (1), the following Equation (2) is obtained.

P1=−B·K·I1·(1−exp(−β·L1))/L1 (2)

In the Equation (2), K represents a constant αt/(Cp·ρ·f).
(Case where Aperture Diameter is DA2)

In the case where the aperture diameter is DA2, the second acoustic-wave measurement value P2, a second light intensity I2, and the second measurement length L2 are substituted for the parameters in the Equation (1). The second acoustic-wave measurement value P2 and the second light intensity I2 are actually measured. The second measurement length L2 is calculated based on the aperture diameter DA2. For example, when the aperture diameter DA2 is 0.13, the second measurement length L2 is 40 μm.

By substituting the second acoustic-wave measurement value P2, the second light intensity I2, and the second measurement length L2 for the parameters in the Equation (1), the following Equation (3) is obtained.

P2=−B·K·I2·(1−exp(−β·L2))/L2 (3)

The operation control unit 150 computes the simultaneous equations of the Equations (2) and (3). It is thereby possible to obtain the bulk modulus B and the light absorption coefficient β of the measurement region within the skin 8.

### (Consideration of β·L)

The method of measuring the elastic modulus described above is effective because the skin 8 contains much water inside. However, for example, the amount of water in an uppermost surface (a horny cell layer) of the skin 8 is about 20%, which is the smallest among the amounts of water within a living organism. Accordingly, the horny cell layer transmits about 99% of the near-infrared laser light having the wavelength of 1.45 μm. In the horny cell layer having such a small amount of water, the generated acoustic wave is small and the elastic modulus measuring apparatus 1 is possibly unable to accurately measure the elastic modulus of the horny cell layer. For example, acoustic waves generated by the water in deeper regions than the horny cell layer possibly affect the acoustic wave from the horny cell layer.

To accurately measure the elastic modulus of a region having a small amount of water such as the horny cell layer, it is necessary to increase a light absorption amount of the region. In the Equation (1), a term indicating light absorption is (1−exp(−β·L)). Therefore, it is important to set (1−exp(−β·L)) as close to 1 as possible.

It is preferable to set a value of β·L equal to or greater than 3 so as to set the light absorption amount to about 95% or more of the laser light. That is, when the value of β·L is equal to or greater than 3, it is possible to consider that the bulk modulus B obtained in the present embodiment is accurate and valid. Furthermore, 95% or more of the laser light can be absorbed in the measurement region when the value of β·L is equal to or greater than 3. Therefore, the transmitted light hardly influences the measurement. Conversely, when the value of β·L is smaller than 3, the bulk modulus B obtained in the present embodiment greatly varies and is not necessarily valid.

Therefore, the operation control unit 150 according to the present embodiment determines that the calculated bulk modulus B is valid when values of β·L1 and β·L2 are equal to or greater than 3. The elastic modulus measuring apparatus 1 can thereby obtain an accurate elastic modulus of the skin 8. Furthermore, the user can determine the accuracy of the calculated elastic modulus to some extent by referring to the values of β·L1 and β·L2.

To obtain the value of β·L equal to or greater than 3 in the horny cell layer, it is considered to use, for example, a quantum-cascade laser or a DFB (Distributed Feedback) laser having a wavelength of 2.7 μm as the light source. When the wavelength of the laser light is about 2.6 μm to 2.8 μm, the light absorption coefficient β exceeds 300 (1/mm). Accordingly, even if the measurement length L is 10 μm (0.01 mm), the value of β·L is equal to or greater than 3. Therefore, the elastic modulus measuring apparatus 1 according to the present embodiment can accurately measure the bulk modulus B of the horny cell layer.

Needless to mention, the measurement length L can be increased so as to set the value of β·L equal to or greater than 3. As described above, the measurement length L can be represented as (the laser light wavelength)/(2·NA²). Therefore, it can be considered to reduce the numerical aperture NA as well as the use of the light source that can ensure the long wavelength of the laser light. The numerical aperture NA is represented as (half of the aperture diameter DA of the aperture adjustment unit 50)/(the focal length of the objective lens 60). Therefore, it suffices to reduce the aperture diameter DA or to increase the focal length of the objective lens 60 so as to reduce the numerical aperture NA.

The elastic modulus measuring apparatus 1 according to the present embodiment measures the acoustic wave generated when the measurement target absorbs the light. That is, the elastic modulus measuring apparatus 1 measures the elastic modulus of the measurement target using the optoacoustic effect. Therefore, the acoustic wave can be generated in a narrow region of the measurement target by focusing the light on the restricted narrow region. The elastic modulus measuring apparatus 1 can thereby measure the elastic modulus of the narrow region of the measurement target. Because the light concentrates in a region corresponding to the focus depth, the elastic modulus can be also measured in a restricted region in a depth direction of the measurement target. The elastic modulus measuring apparatus 1 can thereby measure the elastic modulus precisely also in the depth direction of the skin 8 by measuring the elastic modulus while changing the focal position in the depth direction.

The elastic modulus measuring apparatus 1 does not need to press the measurement target because the elastic modulus measuring apparatus 1 uses the photoacoustic effect. That is, it suffices that the glass substrate 81 of the microphone 80 contacts the measurement target (the skin 8, for example), which does not greatly deform the measurement target.

In the present embodiment, the acoustic wave, which is generated in the measurement region, reaches the microphone 80 via plural layers having different elastic modulus when the measurement region is deep in the skin 8.

When the acoustic wave is propagated through the layers having the different elastic modulus as described above, the elastic modulus measuring apparatus 1 is possibly unable to accurately measure the elastic modulus. Therefore, at the time of measuring the elastic modulus of the measurement region deep in the skin 8 from the surface of the skin 8, the elastic modulus measuring apparatus 1 corrects a measurement result of the elastic modulus measured in the deep measurement target region by converting the elastic modulus measured on the surface of the skin 8 into a certain value. The elastic modulus measuring apparatus 1 can thereby accurately measure the elastic modulus of the measurement region deep in the skin 8.

According to the present embodiment, the surface image of the skin 8 before the skin 8 is irradiated with the light can be compared with that of the skin 8 in a state where the water is adiabatically expanded, using the CMOS image sensor 140. The elastic modulus measuring apparatus 1 can thereby measure the elastic modulus of the measurement region while comparing degrees of expansion before and after the irradiation within the surface of the skin 8. It is thereby possible to observe the surface image of the skin 8 and the corresponding elastic modulus (or the amount of water) within the skin 8.

According to the present embodiment, a database of skin elastic moduli according to ages, sexes, and races can be easily created. This can be used for diagnosis of the skin 8.

In the present embodiment, a material such as gold nanoparticles can be applied into the skin 8 before measuring the elastic modulus so as to increase the adiabatic expansion of the skin 8 and to increase the acoustic wave. The material such as the gold nanoparticles has a characteristic of selectively absorbing light having a specific wavelength. Therefore, it is possible to increase the light absorption within the skin 8 and increase the acoustic wave by applying the material such as the gold nanoparticles into the skin 8.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. An elastic modulus measuring apparatus comprising:
a light source configured to generate light having a wavelength at which a material within a measurement target absorbs the light;
an optical system configured to cause the light to be passed through the measurement target at a desired aperture diameter, and to focus the light on the material;
a detector configured to contact the measurement target and to detect an acoustic wave generated when the material absorbs the light; and
an operation unit configured to calculate an elastic modulus of the material using a first acoustic-wave measurement value obtained in a case where the aperture diameter is a first diameter and a second acoustic-wave measurement value obtained in a case where the aperture diameter is a second diameter different from the first diameter.

2. The apparatus of claim 1, wherein
the operation unit is configured to calculate a first measurement length in the case where the aperture diameter is the first diameter and a second measurement length in the case where the aperture diameter is the second diameter, the first and the second measurement lengths being twice as large as focus depths, and
the operation unit is configured to calculate the elastic modulus of the material and a light absorption coefficient of the material using the first and second acoustic-wave measurement values, the first and second measurement lengths, and first and second light intensities, when it is assumed that an intensity of the light in the case where the aperture diameter is the first diameter is the first light intensity and that an intensity of the light in the case where the aperture diameter is the second diameter is the second light intensity.

3. The apparatus of claim 2, wherein the operation unit is configured to operate simultaneous equations obtained by applying Equation (1) to the case where the aperture diameter is the first diameter and applying the Equation (1) to the case where the aperture diameter is the second diameter, respectively, and
the operation unit is configured to calculate an elastic modulus B of the material and a light absorption coefficient β of the material using the simultaneous equations:
$$P = - B \cdot α t \cdot \frac{{I}_{0} ( 1 - {e}^{- β L} )}{Cp \cdot L \cdot ρ \cdot f} \qquad ( 1 )$$
where P represents the first or second acoustic-wave measurement value, B represents a bulk modulus of the material, αt represents a linear expansion coefficient of a main material mainly constituting the material, I_{O} represents the first or second light intensity, β represents the light absorption coefficient of the material, L represents the first or second measurement length, f represents a pulse frequency of the light, ρ represents a density of the material mainly constituting the material, and Cp represents a specific heat at constant pressure of the material mainly constituting the material.

4. The apparatus of claim 2, wherein the operation unit is configured to determine that the calculated elastic modulus B of the material is valid when both a multiplication of the light absorption coefficient β of the material by the first measurement length L and a multiplication of the light absorption coefficient β of the material by the second measurement length L are equal to or greater than 3.

5. The apparatus of claim 3, wherein the operation unit is configured to determine that the calculated elastic modulus B of the material is valid when both a multiplication of the light absorption coefficient β of the material by the first measurement length L and a multiplication of the light absorption coefficient β of the material by the second measurement length L are equal to or greater than 3.

6. The apparatus of claim 3, wherein
the measurement target is a skin, and
the operation unit is configured to calculate the elastic modulus B of the material and the light absorption coefficient β of the material by approximating the linear expansion coefficient αt of the main material mainly constituting the material to a linear expansion coefficient of water, approximating the density ρ of the material to a density of the water, and approximating the specific heat at constant pressure Cp of the material to a constant pressure specific heat of the water.

7. The apparatus of claim 4, wherein
the measurement target is a skin, and
the operation unit is configured to calculate the elastic modulus B of the material and the light absorption coefficient β of the material by approximating the linear expansion coefficient αt of the main material mainly constituting the material to a linear expansion coefficient of water, approximating the density ρ of the material to a density of the water, and approximating the specific heat at constant pressure Cp of the material to a constant pressure specific heat of the water.

8. The apparatus of claim 1, wherein
the light source is a near-infrared laser diode, and
the optical system comprises:
an aperture adjustment unit configured to cause the light to be passed through the measurement target at a desired aperture diameter;
an objective lens configured to focus laser light from the near-infrared laser diode on the material; and
a focus adjustment unit configured to control the objective lens to operate in order to focus the laser light on the material.

9. The apparatus of claim 8, wherein
the objective lens is configured to cause visible light as well as the laser light to be passed through the measurement target, and
the optical system further comprises:
a focus detection element configured to receive the laser light or the visible light reflected from the measurement target, and to detect whether the laser light is focused on the material; and
an imaging element configured to receive the visible light reflected from the measurement target and to pick up an image of the measurement target.

10. An elastic modulus measuring method comprising:
generating light having a wavelength at which a material within a measurement target absorbs the light;
causing the light to be passed through the measurement target at a desired aperture diameter, and focusing the light on the material;
detecting an acoustic wave generated when the material absorbs the light; and
calculating an elastic modulus of the material in an operation unit using a first acoustic-wave measurement value obtained in a case where the aperture diameter is a first diameter and a second acoustic-wave measurement value obtained in a case where the aperture diameter is a second diameter different from the first diameter.

11. The method of claim 10, wherein
the operation unit calculates a first measurement length in the case where the aperture diameter is the first diameter and a second measurement length in the case where the aperture diameter is the second diameter, the first and the second measurement lengths being twice as large as focus depths, and
the operation unit calculates the elastic modulus of the material and a light absorption coefficient of the material using the first and second acoustic-wave measurement values, the first and second measurement lengths, and first and second light intensities, when it is assumed that an intensity of the light in the case where the aperture diameter is the first diameter is the first light intensity and that an intensity of the light in the case where the aperture diameter is the second diameter is the second light intensity.

12. The method of claim 11, wherein the operation unit is configured to operate simultaneous equations obtained by applying Equation (1) to the case where the aperture diameter is the first diameter and applying the Equation (1) to the case where the aperture diameter is the second diameter, respectively, and
the operation unit is configured to calculate an elastic modulus B of the material and a light absorption coefficient β of the material using the simultaneous equations:
$$P = - B \cdot α t \cdot \frac{{I}_{0} ( 1 - {e}^{- β L} )}{Cp \cdot L \cdot ρ \cdot f} \qquad ( 1 )$$
where P represents the first or second acoustic-wave measurement value, B represents a bulk modulus of the material, αt represents a linear expansion coefficient of a main material mainly constituting the material, I_{O} represents the first or second light intensity, β represents the light absorption coefficient of the material, L represents the first or second measurement length, f represents a pulse frequency of the light, ρ represents a density of the material mainly constituting the material, and Cp represents a specific heat at constant pressure of the material mainly constituting the material.

13. The method of claim 11, wherein the operation unit is configured to determine that the calculated elastic modulus B of the material is valid when both a multiplication of the light absorption coefficient β of the material by the first measurement length L and a multiplication of the light absorption coefficient β of the material by the second measurement length L are equal to or greater than 3.

14. The method of claim 12, wherein the operation unit is configured to determine that the calculated elastic modulus B of the material is valid when both a multiplication of the light absorption coefficient β of the material by the first measurement length L and a multiplication of the light absorption coefficient β of the material by the second measurement length L are equal to or greater than 3.

15. The method of claim 12, wherein
the measurement target is a skin, and
the operation unit is configured to calculate the elastic modulus B of the material and the light absorption coefficient β of the material by approximating the linear expansion coefficient αt of the main material mainly constituting the material to a linear expansion coefficient of water, approximating the density ρ of the material to a density of the water, and approximating the constant pressure specific heat Cp of the material to a specific heat at constant pressure of the water.

16. The method of claim 13, wherein
the measurement target is a skin, and
the operation unit is configured to calculate the elastic modulus B of the material and the light absorption coefficient β of the material by approximating the linear expansion coefficient αt of the main material mainly constituting the material to a linear expansion coefficient of water, approximating the density ρ of the material to a density of the water, and approximating the specific heat at constant pressure Cp of the material to a constant pressure specific heat of the water.
